(19) United States
(12) Patent Application Publication
(10) Pub. No.: US 2011/0060194 A1
(43) Pub. Date: Mar. 10, 2011

(54) RETRACTOR

(76) Inventors: Olof Risto, Linkoping (SE); Stefan Lind, Jonkoping (SE)

(21) Appl. No.: 12/876,987

(22) Filed: Sep. 7, 2010

Related U.S. Application Data
(63) Continuation-in-part of application No. 11/722,879, filed on Jun. 26, 2007.

(30) Foreign Application Priority Data
Dec. 29, 2004 (SE) 0403211-6
Aug. 26, 2010 (SE) 1000868-8

(51) Int. Cl. A61B 1/32

(52) U.S. Cl. 600/210

(57) ABSTRACT

A retractor for placement in a wound to hold the wound open during a surgical procedure comprising a closed loop formed of a plastic material permeable to X-ray radiation, said closed loop having two opposite members that can be brought together from a starting position to enable insertion of wound edge gripping elements of the retractor into the wound and thereafter through resilience return towards the starting position and brought in contact with the edges of the wound and hold the wound edges apart and in a self-holding way retain the retractor in the wound.

### CROSS REFERENCE TO RELATED APPLICATION

This application is a continuation-in-part of U.S. patent application Ser. No. 11/722,879 filed Jun. 26, 2007, the disclosure of which is hereby incorporated by reference. This application also claims partial priority from Swedish Patent Application Serial No. 1000868-8, filed Aug. 26, 2010.

### FIELD OF THE INVENTION

The present invention relates generally to a retractor, which is intended to be placed in a wound in order to hold this open during a surgical operation.

### BACKGROUND OF THE INVENTION

A known retractor of this kind comprises a curved hook of metal that is inserted into a wound (e.g., a surgical incision) and pulled laterally against a wound edge in order to hold the wound open to facilitate a surgical operation. Advantageously two retractors are used, which are pulled in opposite directions in order to hold the wound as open as possible. A drawback with such a retractor is that it requires a person holding it during the entire operation. Another drawback is that the retractor, during an X-ray examination, may block out for instance a fractured bone that is to be X-rayed, thus necessitating removal of the retractor during the examination. A further drawback is that the retractor must be subjected to an extensive cleaning when it is washed, sterilized and repacked before it can be used in a new surgical operation.

Thus, a retractor that does not need to be held during an operation, that does not block X-rays and that does not require extensive and time consuming cleaning before each use would be very desirable to overcome some of the most limiting characteristics of the prior art instruments.

### SUMMARY OF THE INVENTION

The present invention is directed to a retractor that overcomes some or all of the above-mentioned disadvantages of the prior art instruments.

In one aspect, the disclosure provides a retractor characterized in that it is constituted of a shackle of a plastic material that is radiolucent (medical term meaning pervious to X-rays), which has two resilient legs that in a springy way can be brought together from a starting position in order to be inserted into the wound or cut and thereafter through resilient return towards the starting position be brought to contact with the edges of the wound, thereby holding the wound edges apart and in a self-supporting way fasten the retractor in the wound.

Since the retractor is made of a radiolucent plastic material pervious to X-rays it can remain in the wound during radiolucent or fluoroscopic X-ray examinations without blocking the visibility of, for instance, a bone fracture.

The retractor is made of plastic material and intended to be used as a single use product, which makes it hygienic and which means that no extensive and costly cleaning work must be carried out.

The two resilient legs of the retractor are pressed out against the edges of the wound when the retractor is placed in the wound and make the retractor self-retaining, thereby eliminating the need to hold the retractor during the surgical operation once it has been placed in the wound.

Since each leg in its free end may have an outwards directed, hook-like protrusion for gripping a wound edge the retractor may be better located in the wound.

The legs of the retractor preferably have a knee like bend, the bends being located opposite each other. This means that a larger space is obtained above the wound, which facilitates the surgical operation.

The shackle of the retractor is preferably made in one piece which simplifies the fabrication process.

Radiolucent material in accordance with the invention may be used for a variety of types of retractors that are used to hold apart tissues and parts of the body during surgical operations. For example, the type of retractors that are used to hold muscle and sinew tissues at a distance from bones that are to be worked, and then in particular on the bottom side. Since it becomes possible to X-ray with the retractors in place one can better and more easily than before secure that the bone on the off side is free so that the muscles do not risk to be injured at the working of the bone.

A further advantage with the retractor according to the invention is that it is light in relation to its ability to hold a wound open. This means that you may move the wound in relation to the area beneath that is subject to the operation, which in turn can allow the surgeon to use smaller incisions to optimize the surgeon's angle for the surgery.

The retractor will since it is light remain in place even if you move or turn for instance a limb improving the working conditions of an operation and often shortening the required time and improving the quality at the same time.

It is also easy to adjust the orientation and/or location if needed. Likewise it is an easy step to change to another size, an important factor to increase accuracy and reduction of the patient's and surgeon's time.

In another aspect, the disclosure provides a retractor for placement in a wound to hold the wound open during a surgical procedure comprising a loop formed of a radiolucent plastic material, said loop having two opposite members that can be brought together from a starting position to enable insertion of the retractor into the wound and thereafter through resilience return towards the starting position and brought in contact with the edges of the wound and hold the wound edges apart and in a self-holding way fasten the retractor in the wound.

The retractor may have one or more wound gripping members on the two opposite members.

The loop may comprise a first and second shackle, each of said shackle having two legs, each leg having proximal and distal ends, wherein the legs of each shackle are joined at their proximal ends at a spring head, and wherein the distal ends of the legs of said first shackle are coupled to the distal ends of the legs of said second shackle, said coupled legs of said first and second shackles thereby forming said two opposite members.

In another aspect, the loop may form an oval shape having two long sides, wherein the long sides form the two opposite members.

Accordingly, several advantages of the disclosure are that the retractor is self-holding, radiolucent, lightweight, provides greater space above a wound, and does not require extensive and time consuming cleaning before each use. Still further objects and advantages will become apparent from the detailed description and accompanying drawings.

The features, functions and advantages that have been discussed can be achieved independently in various embodiments of the present disclosure or may be combined in yet other embodiments, further details of which can be seen with reference to the following descriptions and drawings.

### BRIEF DESCRIPTION OF THE DRAWINGS

Further features and advantages of the invention will be seen from the following detailed description, taken in conjunction with the accompanying drawings, wherein like numerals depict like parts, and wherein:

FIG. 1 is a frontal view illustrating a retractor in accordance with the present disclosure in a starting position.

FIG. 2 is a frontal view illustrating the retractor of FIG. 1 in a compressed position.

FIG. 3 is a frontal view illustrating another embodiment of a retractor in accordance with the present disclosure.

FIG. 4 is a top view illustrating another embodiment of a retractor in accordance with the present disclosure.

FIG. 5 is a top view illustrating yet another embodiment of invention retractor in accordance with the present disclosure.

FIG. 6 is a top view illustrating yet another embodiment of invention retractor in accordance with the present disclosure.

FIG. 7 is a top view illustrating of the embodiment shown in FIG. 6 but in another state.

### DETAILED DESCRIPTION OF PREFERRED EMBODIMENTS

In the following description, reference is made to the accompanying drawings, which form a part hereof, and in which is shown, by way of illustration, various embodiments of the present disclosure. It is understood that other embodiments may be utilized and changes may be made without departing from the scope of the present disclosure.

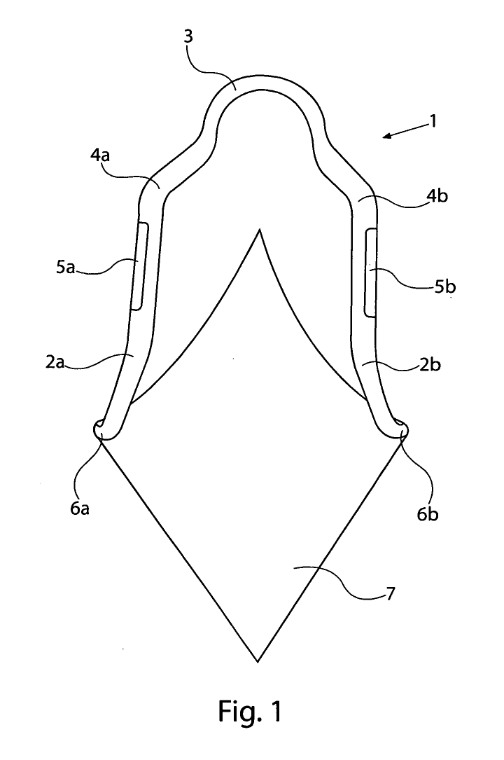

FIG. 1 illustrates a primary example of a retractor provided by this disclosure. The retractor 1 is constituted of a shackle that has two elongate, resilient legs 2a, 2b and an essentially part circular spring head 3, from which the legs 2a, 2b extend. The retractor 1 is made of one piece and of a radiolucent plastic material, preferably environment friendly decomposable hard plastic.

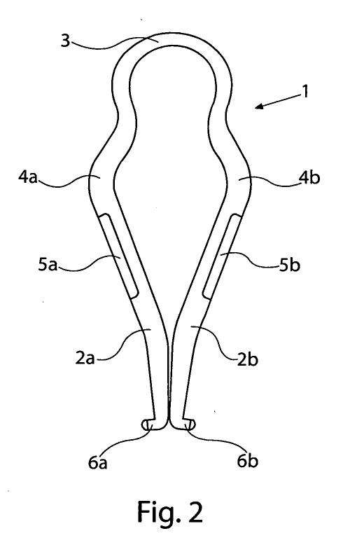

The two legs 2a, 2b are in a resilient way possible to be brought together from a starting diverging position (FIG. 1), in which there is a distance between the lower parts of the legs 2a, 2b, to an entirely compressed position (FIG. 2), in which the legs 2a, 2b are in contact with each other in their lower parts.

The legs 2a, 2b may have a knee like bend 4a, 4b. The bends 4a, 4b may be located opposite each other. In the proximity of each bend 4a, 4b a grip 5a, 5b may be arranged. The grips 5a, 5b may have an uneven surface structure.

Each leg 2a and 2b may further have in their free ends an outwards directed hook-like protrusion 6a, 6b that may be a short extension of each leg 2a, 2b.

The retractor 1 may be inserted into a wound 7, in order to hold this open during a surgical operation.

When the retractor 1 is to be inserted into the wound 7 the legs 2a and 2b are pressed together (FIG. 2) by the user pushing the grips 5a, 5b towards each other so that the lower parts of the legs 2a, 2b are brought closer to, or in contact with, each other. Thereafter, the retractor 1 may be inserted into the wound 7. When the user releases the pressure on the grips 5a, 5b the legs 2a, 2b are once again separated by resilience and contact the edges of the wound 7. The resilient return of the legs 2a, 2b towards the starting position results in the edges of the wound 7 being held apart. The inherent resilience of the retractor 1 causes the retractor 1 to be seated in the wound 7 in a self-retaining manner.

The grips 5a, 5b facilitate the compression of the legs 2a, 2b, since they help the user to get a steady grip on the retractor 1. The grips 5a, 5b also reduce the risk for the user to slip and injure the edges of the wound 7.

The outwards directed, hook-like protrusions 6a, 6b may be brought in contact with the edges of the wound 7 when the retractor is placed in the wound 7 and result in a safe seating of the retractor in the wound 7.

Below the resilient head 3 of the retractor 1 and between the bends 4a, 4b a space is formed, which provides space for the hands of the user and instruments that may be used during the surgical operation, thereby facilitating execution of the surgical operation.

Within the frame of the inventive thought one can also consider other embodiments of the retractor than the one described above. For instance, the legs of the retractor may be straight instead of being bent outwards in the ends. One can further consider the legs being bent more or less downwards a distance from the hooks or tips, depending on what they are intended to be used for. The legs may be straight or, as in FIG. 1, bent slightly outwards. Moreover, the tips may be considerably longer than depicted.

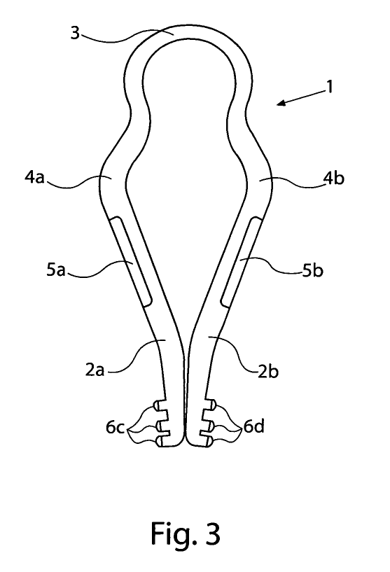

Referring now to FIG. 3, in order to achieve a gentler and a more elongate separation of the wound edges, the retractor may be provided with a number of parallel downwards extending hooks 6c, 6d, approximately as a comb or fence. Each tooth in the lower end may be provided with one or several outward directed points. The resilient force thus may be distributed to several points and, since the wound edges can be held apart over a greater length, the separation in the middle does not need to be as large for the same operation. Furthermore, the use of retractors of this type allows one to separate the wound and execute the surgical operation with fewer retractors than required by the prior art instruments, which improves the accessibility to the operation area. If so desired, each comb tooth or hook may be slightly resilient in order to secure an even force distribution. The points in the end of the retractor may also have different lengths depending on the field of application.

The shape, location and number of the of the wound edge gripping elements can vary depending on the intended use. For instance they may take the shape of an elongate straight of curved profile gripping under the wound edge.

Furthermore, a retractor in accordance with the present disclosure may be provided with grip parts extending above the plane of the retractor and that may be grooved and/or concave to reduce the risk of the device slipping from one's grip, and avoiding the need to use valuable time to get a good grip.

A suitable material for the retractor in accordance with the present disclosure may include polyamide or polyoxymethylene, which easily can be destructed through burning.

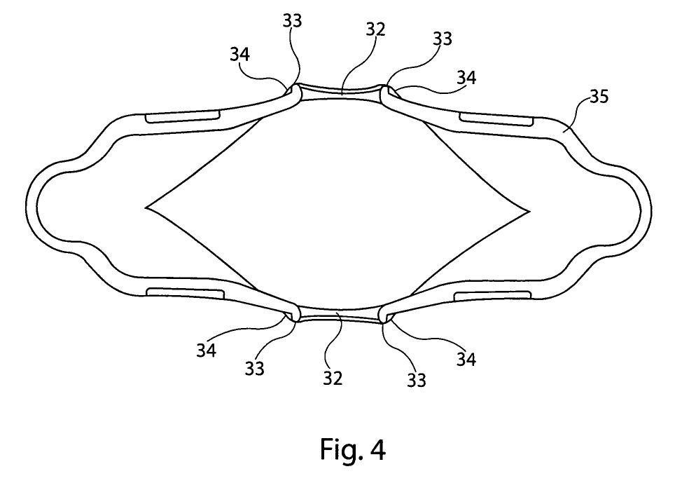

FIG. 4 illustrates a further example of a retractor provided by the present disclosure. The retractor essentially consists of two retractors, as depicted in FIGS. 1 and 2, facing each other and joined in their outer ends of the shackle legs 35 by connecting pieces 32 to form a closed loop. Four gripping points 33 are provided on fingers 34. The connecting pieces 32 are rather flat and flexible, serving as hinges between the two symmetric flange halves that thus can be mounted separately.

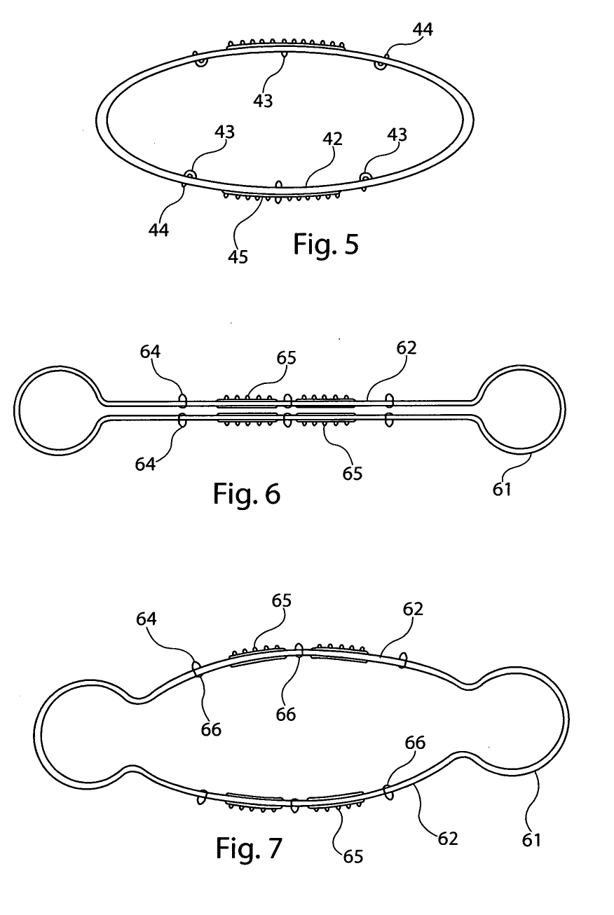

FIG. 5 illustrates yet another example of a retractor provided by the present disclosure. The retractor shown in FIG. 5 has an oval shape and three gripping points 44 are distributed along each long side 42 of the retractor. Each gripping point is suspended in a resilient finger 43. Centrally located on the outside of each long side, a grip part 45 is arranged for the surgeon's fingers when the long sides of the retractor are pressed towards each other when the retractor is inserted into a wound.

An essential advantage of such closed loop structures as provided by FIGS. 4 and 5 is the increase in force that is achieved with a still very light device. With such a structure, not only the ends of the retractor will serve as widening springs, but the entire loop may provide resilient force and may thus work as a spring. Since the ends of the retractor can be supported against the operated body at the ends of the wound a very stable retractor is achieved that is also minimally in the way of the surgeon.

Yet another example of a closed loop retractor is shown in FIG. 6 and FIG. 7. This retractor has two long sides 62 that are slightly curved connected in there ends by omega shaped parts 61. Gripping points 64 at the ends of curved fingers 62 and grip parts 65 are arranged on the two long sides. The long sides and the omega parts are dimensioned so that when the force exerted by the user is sufficient to straighten the long sides also the omega shaped parts will become essentially closed. In FIG. 7 the retractor is free and in FIG. 6 it is compressed and ready for insertion into a wound or incision. Instead of arranging the gripping points precisely opposite each other they may be slightly displaced relative each other allowing the gripping point to form one line facilitating the insertion into the wound or incision, This is of course also applicable for the other retractors according to the invention.

The gripping points need not necessarily all be sharp. Some of the gripping points (perhaps all but one on each side) may be more blunt or elongated in a direction basically in parallel with the length direction of the retractor, allowing them to slide along the wound edge when the wound is dilated thus reducing the strain on skin and other tissues that are to be kept apart.

In the embodiment depicted by FIG. 5, four gripping point are used on each side but the retractor may instead be provided with only one, or some other number on each long side. Provided that the long sides of the retractor are resilient enough these sides may when pressed against each other straighten so that the wound edge gripping elements are located in line when they are to be inserted into an incision.

The retractors according to the present disclosure, both in an open U-shaped structure as demonstrated by FIGS. 1-3 and in a closed oval-shaped structure as demonstrated by FIGS. 4 and 5, will be close to the body and out of the way for the surgeon. Both the open and closed versions of the invented retractor are flexible and dynamically opens and follow the wound at dissection of the tissue, movement of the body part or extending in the length direction of the wound.

Since the retractors may be constructed of plastic in a single press operation as one unit or body, efficient production and destruction is possible. As such, a retractor in accordance with the present disclosure may be disposable or single use, which results in considerable savings in logistic work compared to prior art retractors that have to be sterilized prior to each use.

1. A retractor for placement in a wound to hold the wound open during a surgical procedure comprising a loop formed of a plastic material permeable to X-ray radiation, said loop having two opposite members, each having or including at least one wound edge gripping element, said two opposite members can be brought together from a separated starting position to enable insertion of the gripping elements of the retractor into the wound and thereafter through resilience return towards the starting position in contact with the edges of the wound holding the wound edges apart and in a self-retaining way fastening the retractor in the wound.

2. The retractor of claim 1, wherein the loop is open and has the shape of a shackle and the two opposite members are the legs of the shackle and the outer ends of said legs can be brought together for insertion into a wound of said wound edge gripping elements.

3. The retractor of claim 2, wherein the legs each have a bend such that the space between the legs at a position distal to the bend is substantially greater than the space between the legs at a position proximal to the bend when the retractor is in the starting position.

4. The retractor of claim 2, wherein said wound edge gripping member comprises an outward directed hook-like protrusion at the distal end of each leg.

5. The retractor of claim 2, wherein the shackle is made in one piece.

6. The retractor of claim 2, wherein the legs are bent downwards a distance from their distal ends.

7. The retractor of claim 2, wherein the legs of the retractor at their distal ends are provided with a row of downward extending thin pins that in the lower ends are provided with one or several outward facing points.

8. The retractor of claim 2, wherein the legs are provided with upwards protruding grip parts.

9. The retractor of claim 2, wherein the plastic material comprises a decomposable hard plastic.

10. The retractor of claim 2, wherein the plastic material comprises polyoxymethylene.

11. The retractor of claim 1, wherein the loop is closed.

12. The retractor of claim 11, wherein the loop comprises a first and second shackle, each of said shackles having two legs, each leg having proximal and distal ends, wherein the legs of each shackle are joined at their proximal ends at a spring head, and wherein the distal ends of the legs of said first shackle are coupled to the distal ends of the legs of said second shackle, said coupled legs of said first and second shackles thereby forming said two opposite members.

13. The retractor of claim 12, wherein the legs each have a bend such that the space between the opposing legs at a position distal to the bend is substantially greater than the space between the legs at a position proximal to the bend when the retractor is in the starting position.

14. The retractor of claim 12, wherein said wound edge gripping member comprises an outward directed hook-like protrusion at the distal end of each leg.

15. The retractor of claim 12, wherein the legs each have a knee like bend, wherein the bends are located opposite each other.

16. The retractor of claim 12, wherein the shackles are made in one piece.

17. The retractor of claim 12, wherein the legs are bent downwards a distance from their distal ends.

18. The retractor of claim 12, wherein the legs of the retractor at their distal ends are provided with a row of downward extending thin pins that in the lower ends are provided with one or several outward facing points.

19. The retractor of claim 11, wherein the plastic material comprises a decomposable hard plastic.

20. The retractor of claim 11, wherein the plastic material comprises polyoxymethylene.

21. The retractor of claim 12, wherein the legs are provided with upwards protruding grip parts.

22. The retractor of claim 12, wherein the distal ends of the legs of said first shackle are coupled to the distal ends of the legs of said second shackle via connecting pieces.

23. The retractor of claim 22, wherein the connecting pieces are bendable in order to serve as hinges.

24. The retractor of claim 11, wherein the loop forms an oval shape having two long sides, wherein said long sides form said two opposite members.

25. The retractor of claim 11, wherein the loop is made in one piece.
